(19) United States
(12) Patent Application Publication
(10) Pub. No.: US 2024/0139414 A1
(43) Pub. Date: May 2, 2024

(54) Medical Flow Sensor Module Assembly

(71) Applicant: RAUMEDIC AG, Münchberg (DE)

(72) Inventors: Lena PIESCHNIK, Bindlach (DE); Susan ZEININGER, Untersteinach (DE); Stefan WEISS, Hof (DE); Bastian RAMMING, Untersteinach (DE); Ricardo EHRENPFORDT, Zwickau (DE); Thomas RUHLAND, Bayreuth (DE)

(73) Assignee: RAUMEDIC AG, Münchberg (DE)

(21) Appl. No.: 18/273,260

(22) PCT Filed: Jan. 19, 2022
(86) PCT No.: PCT/EP2022/051123

§ 371 Date: Jul. 19, 2023

(30) Foreign Application Priority Data
Jan. 20, 2021 (DE) 10 2021 200 496.2

(51) Int. Cl. A61M 5/168; A61M 5/14; A61M 5/36; G01F 1/667

(52) U.S. Cl. CPC A61M 5/16886; A61M 5/1413; A61M 5/365; G01F 1/667; A61M 2205/3334; A61M 2205/3375

(57) ABSTRACT

A medical flow sensor module assembly is part of an installation for patient monitoring and/or patient care. A single-use sensor module has a channel body with a flow channel A flow sensor is used to measure a fluid flow through the flow channel A flow sensor interface is in signal connection with the flow sensor. A reusable housing module of the module assembly serves to accommodate the sensor module. The housing module has at least one electronics unit and an electronics unit interface via which the electronics unit is in signal connection with the flow sensor interface. The housing module has at least two housing sections which enclose the sensor module in a form-fitting manner. This results in an assembly that can be used cost-efficiently.

### CROSS-REFERENCES TO RELATED APPLICATIONS

This application claims the priority of German Patent Application, Serial No. DE 10 2021 200 496.2, filed Jan. 20, 2021, the content of which is incorporated herein by reference in its entirety as if fully set forth herein.

### FIELD OF THE INVENTION

The invention relates to a medical flow sensor module assembly.

### BACKGROUND OF THE INVENTION

A sensor module for such a module assembly is known from EP 2 107 347 B1. WO 2019/034 570 A1 discloses a sensor assembly comprising a pressure sensor and a thermal gas sensor. US 2011/0 118 705 A1 discloses a sensor and control unit for fluid flow control and a method for controlled fluid delivery. U.S. Pat. No. 3,921,622 discloses a method and apparatus for ultrasonic detection of inclusions in a flowing fluid. DE 10 2012 111 757 A1 discloses a device for ultrasonic flow measurement in hose and/or plastic pipe systems.

### SUMMARY OF THE INVENTION

It is an object of the present invention to provide a medical flow sensor module assembly that can be used in a cost-efficient manner.

This object is achieved according to the invention by a module assembly having the features described hereinafter.

According to the invention, it has been recognized that a modular division of the module assembly on the one hand into a single-use sensor module (disposable), comprising in particular a flow sensor, and on the other hand into a reusable housing module (reusable) creates the possibility of accommodating in particular expensive electronics components in the reusable housing module, which can be used again after replacing the single-use sensor module. This increases the cost efficiency of the module assembly, as the reusable housing module can be used with a plurality of sensor modules, and possibly with a multitude of sensor modules used in succession. The form-fit of the sensor module in the housing module ensures in particular that the sensor module is secured against rotation. The sensor module is positioned in a defined manner due to the form-fit, so that a connection between supply components and components to be supplied between the modules is also possible via correspondingly aligned connection interfaces, in particular via plug connectors.

The electronics unit of the sensor module may have a data processing component and/or a memory component and/or a sensor control component and/or an output component. The electronics unit may have a microcontroller. The electronics unit may have adjustment and calibration curves stored therein. These curves may serve to match flow rates with the particular fluidic materials to be measured. The electronics unit can be equipped with software that can be used to process algorithms for feature extraction, selection and classification. The software may include a reminder function, in particular a reminder of a need to replace the single-use sensor module. The interfaces of the module assembly can be designed as possibly sealed plugs. A power supply and/or a serial bus signal can be transmitted via the interfaces of the module assembly. Alternatively or additionally, the single-use sensor module can be supplied with energy via a battery or an accumulator. RS232, RS485 and CAN can be used as interface or bus standards. The interfaces of the module assembly can be designed to be wireless. The standards Bluetooth or Wifi can be used. The electronics unit can have a possibly inductively operating interface for signal connection with an external device. This can be used for data evaluation and/or data display. The module assembly can be designed such that telemetric monitoring of the function and the measured values of the module assembly is possible in cooperation with the external device. Data processing or data evaluation of measured values of the module assembly can be performed via an app and/or via a cloud solution. The module assembly can have means for plagiarism protection, so that in particular the use of an unauthorized sensor module in the module assembly is prevented. Such protection against plagiarism can be achieved by ID recognition of identification data, in particular of the sensor module. The module assembly may be designed in the form of a connector that can be used in particular between tubing sections of the installation for patient monitoring and/or patient care. The module assembly can have Luer connections for coupling to external fluid-carrying components of the installation for patient monitoring and/or patient care.

The reusable housing module may have a unit for recording a signal for collecting a duration of use of the single-use sensor module. In particular, a number of disposables used sequentially with the reusable may be recorded.

The electronics unit has at least one data processing and control component for controlling the flow sensor and for recording, processing and transmitting sensor signals. Such a design of the electronics unit enables flexible use of the module assembly.

This applies in particular to a module assembly in which the two housing sections each comprise an electronics unit.

An electrical connector between the housing sections to form a signal connection between the at least two electronics units is cost-efficient. The plug-in connection can be designed as a sealed plug-in connection.

A hinged connection of the housing sections by means of a swivel joint is easy to handle and enables a cost-efficient execution of a form fit of the housing sections around the sensor module.

A fixation of the housing sections to one another in a folded connection position prevents an unintentional separation therefrom. The fixation can be implemented by a snap-in connection of the housing sections with each other. When engaging, a defined clicking sound can indicate to the user a correct closure of the housing module. The fixation can have at least one latching element which prevents an unintentional loosening of the fixation.

The housing module can have an indicator unit for emitting a signal when the folded connection position is fully reached and/or when the folded connection position is not fully reached.

At least two sensors, one of which is the flow sensor of the sensor module, increase the application possibilities of the module assembly. Several single-use sensors can be used. In addition to a flow sensor, for example, a pressure sensor, a temperature sensor or an acceleration sensor or a combination of these sensors can be used.

A sensor embodiment in which one of the sensors is part of the reusable housing module as a reusable sensor increases a cost efficiency of the module assembly. Sensors that are not contaminated during use may be part of the reusable housing module.

An ultrasonic sensor can be used for bubble detection in the flow channel Examples of such ultrasonic sensors can be found in EP 2 813 845 B 1, DE 102 09 254 B4, DE 102 09 255 B4 and US 2013/305839A1. Such an ultrasonic sensor can be used for bubble reduction in the flow channel.

A multi-part design of the channel body such that the flow channel has a plurality of sequential channel sections which are formed by different channel body sections further extends the application possibilities of the module assembly. The module assembly may have a plurality of sensors.

Each of the multiple channel body sections can be used by at least one of the sensors as a measuring section for measuring the fluid flowing through.

If the module assembly has several sensors, each channel section can be used by at least one of the sensors as a measuring section for measuring the fluid flowing through. The channel sections can be connected to each other via a snap-in connection.

An indicator unit of the housing module can be digital, optical and/or acoustic. The indicator unit can be designed to issue a warning signal, in particular when a measured value limit is exceeded. The indicator unit can carry out a comparison of a predefined target measured value with a current actual measured value. The indicator unit can be designed to emit tones, colors or also as a vibration indicator. The indicator unit can be configured to preselect a fluid medium to be measured.

A reusable pump unit for generating a flow of media through the flow channel may provide for a self-sufficient module assembly in case of an emergency. For example, such a pump unit may also serve to stimulate a lack of or insufficient media flow through the module assembly initially or during operation. The pump unit may also be used to assist or stimulate fluid flow that would otherwise be achievable by gravity. In certain applications, the pump unit may also eliminate the need for a pump that would otherwise be provided at the installation for patient monitoring and/or patient care. The pump unit may be controllable by the electronics unit of the reusable housing module.

Examples of embodiments of the invention are explained in more detail below with reference to the drawings.

### BRIEF DESCRIPTION OF THE DRAWINGS

FIG. 1 shows a perspective view of a closed medical flow sensor module assembly as part of an installation for patient monitoring and/or patient care;

FIG. 2 shows the module assembly according to FIG. 1, wherein one of two housing sections, which in the closed position according to FIG. 1 enclose a sensor module of the module assembly in a form-fitting manner, is folded open into an open position;

FIG. 3 shows a schematic longitudinal section through an embodiment of the module assembly, wherein a flow channel with opposite orientation compared to FIG. 2 is illustrated;

FIG. 4 shows a section according to line IV-IV in FIG. 3;

FIG. 5 in a representation similar to FIG. 3, shows a further embodiment of a flow sensor module assembly which, in comparison to the embodiment according to FIGS. 1 to 4, is designed without an ultrasonic sensor in addition to a flow sensor; and

FIG. 6 shows a sectional view of the module assembly according to FIG. 5 similar to FIG. 4.

### DESCRIPTION OF THE PREFERRED EMBODIMENTS

A medical flow sensor module assembly 1 is part of an installation for patient monitoring and/or patient care. The module assembly 1 has a reusable housing module 2, also referred to as a reusable. The housing module 2 has two housing sections, namely a base section 3 and a cover section 4, which are hingedly connected to each other about a hinge axis 8 via a hinge 5 with hinge sections 6, 7.

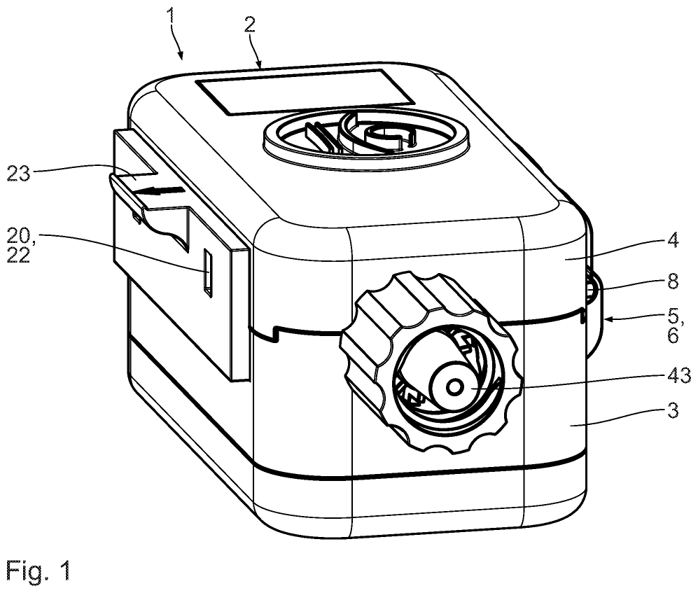

In a closed position, which is also referred to as the folded connection position and is shown in FIG. 1, the two housing sections 3, 4 enclose a single-use sensor module 9 in a form-fitting manner. This form-fitting enclosure serves on the one hand to mechanically hold the sensor module 9 in the folded connection position and also serves to prevent at least selected components of the sensor module from rotating about a longitudinal axis 10 of the sensor module 9, which longitudinal axis 10 is defined by a flow channel 10a of a channel body 11 of the sensor module 9.

The single-use sensor module 9 is also referred to as a disposable.

The sensor module 9 has a flow sensor 12 for measuring a fluid flow through the flow channel 10a. Such a flow sensor is known, for example, from EP 2 107 347 B1.

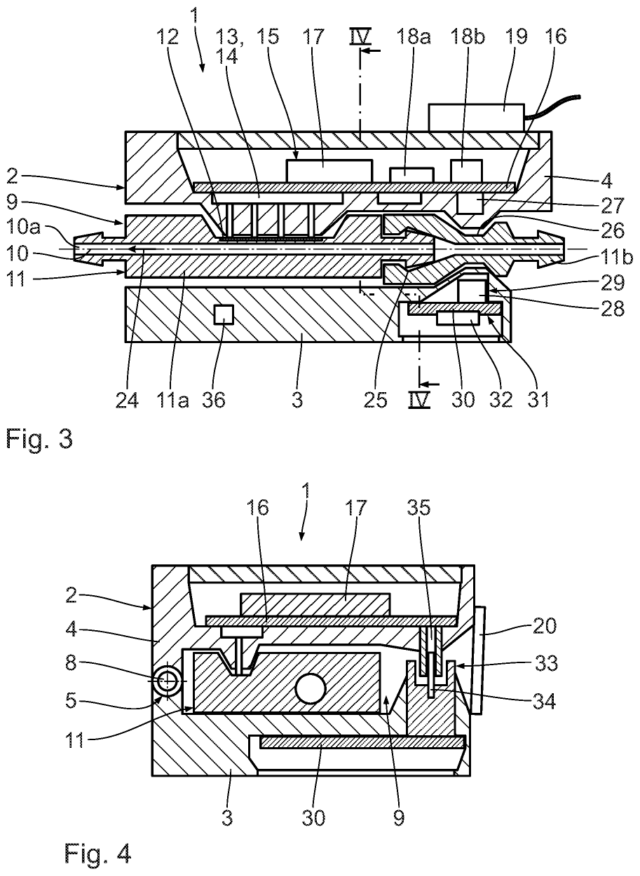

Furthermore, the sensor module 9 has a flow sensor interface 13 (cf. FIG. 3), which is in signal connection with the flow sensor 12. An electronics unit interface 14 is in signal connection with the flow sensor interface 13. Via the electronics unit interface 14, an electronics unit 15 of the housing module 2 is in signal connection with the flow sensor 12.

The two interfaces 13, 14 are designed as electrical plug-in connectors between the housing module 2, i.e. the reusable, and the sensor module 9, i.e. the disposable.

The electronics unit 15 is accommodated in one of the two housing sections 3, 4, namely in the housing cover section 4. The electronics unit 15 has several electronic components which are accommodated on a common circuit board 16 in the housing cover section 4. These components include, among others, a data processing and control component 17, a data storage component 18 and components 18a, 18b for receiving, processing and transmitting sensor signals.

The electronics unit 15 or the module assembly 1 further has an interface 19 for signal connection with an external device. This interface 19 can be designed as an inductive interface. Via the interface 19, a data evaluation as well as a signal and/or data display can be carried out via the external device. Telemetry monitoring of the module assembly is also possible via the interface 19. Depending on the design, the interface 19 can be controlled via an app or cloud application.

The electronics unit 15 may comprise means for plagiarism protection as well as means for ID recognition of the single-use sensor module 9.

The two housing sections 3, 4 of the housing module 2 are fixed to each other in the folded connection position according to FIGS. 1, 3 and 4. This is achieved by means of a snap-in connection 20.

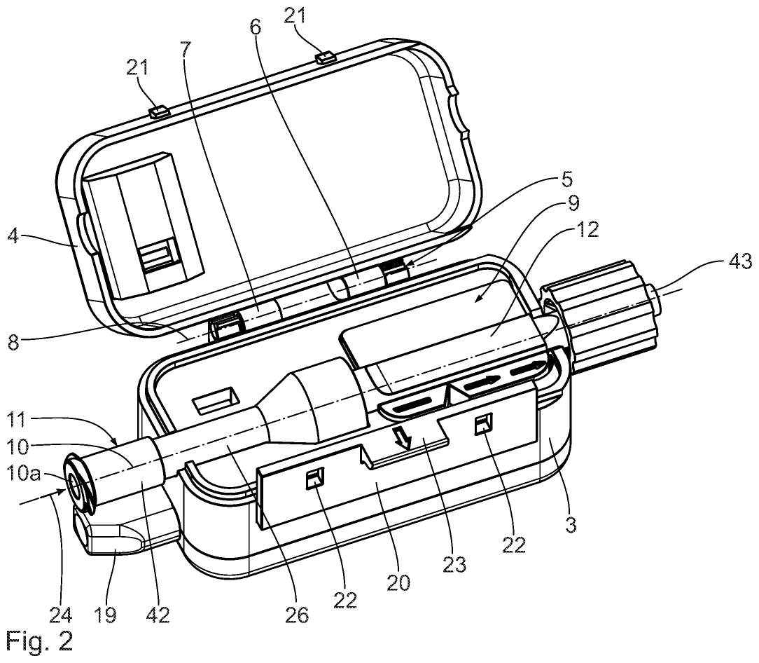

The snap-in connection 20 has latching lugs 21 which are attached to one of the two housing sections 3, 4 and, in the folded connection position, cooperate in a latching manner with latching tabs or latching grooves 22 which are designed in the other one of the two housing sections 4, 3. In the embodiment according to FIGS. 1 and 2, the latching lugs 21 are arranged at the housing cover section 4 and the latching tabs 22 at the housing base section 3.

To facilitate overcoming the latching force of the snap-in connection 20, a projecting actuation section 23 is formed on the housing base section 3. Pressure on the actuation section 23 in FIG. 1 from above causes the latching tabs 22 to lift off and come free from the associated latching lugs 21, so that the housing cover section 4 can be swung open from the folded connection position into the open position as shown in FIG. 2. In order to facilitate the release of the housing cover section 4 when the actuation section 23 is actuated, the housing cover section 4 can be pretensioned in the folded connection position such that it swings open at least a little way into the open position after the latching tabs 22 have been disengaged from the latching lugs 21.

The channel body 11 is made of several parts, namely two parts, and accordingly has two sequential channel sections which are formed by different channel body sections 11a and 11b. In the downstream channel body section 11a in a flow direction 24, the flow sensor 12 for measuring the fluid flow through the flow channel 10a is arranged. Upstream of this channel body section 11a, the further channel body section 11b is arranged, which is tightly connected to the following channel body section 11a via a snap-in connection 25. In the region of the channel section formed by the leading channel body section 11b, this channel body section 11b is configured with a narrowing section 26. The narrowing section 26 may be in the form of an outer circumferential groove. In the schematic illustration according to FIG. 3, this groove is shown to be axially less extended than in the illustration according to FIG. 2.

In the region of the narrowing section 26, two sensor components 27, 28 of a reusable ultrasonic sensor 29 interact with the fluid flowing through the flow channel 10a, in particular for the detection of entrained bubbles in the fluid. Ultrasonic sensors that are suitable for this measuring task are known from DE 102 09 254 B4, DE 102 09 255 B4, U.S. Pat. No. 9,016,137 B and, for example, EP 2 813 845 B1.

The ultrasonic sensor is a reusable sensor and is part of the housing module 2.

The ultrasonic sensor component 27 is mounted on that side of the circuit board 16 of the electronics unit 15 which faces the narrowing portion 26 of the channel body portion 11b. The ultrasonic sensor component 27 is thus accommodated in the housing cover section 4. The ultrasonic sensor component 27 is controlled by the DV control component 17 of the electronics unit 15.

The ultrasonic sensor component 28 is accommodated in the housing base section 3 and also faces the narrowing section 26 of the channel body section 11b. The narrowing section 26, and thus the section of the flow channel 10a bounded thereby, is thus located between the two ultrasonic sensor components 27, 28.

The ultrasonic sensor component 28 accommodated in the housing base section 3 is arranged on a circuit board 30 of a further electronics unit 31 that is accommodated as a whole in the housing base section 3. This circuit board 30 carries an additional data processing component 32, which in particular has the function of controlling the ultrasonic sensor component 28. Other functions explained above in connection with components 17, 18, 18a, 18b can also be performed by the data processing component 32.

The two electronics units 15, 31 are in signal connection with each other via an electronics unit interface. This electronics unit interface is designed as an electrical plug-in connection 33 (cf. FIG. 4). In the folded connection position of the two housing sections 3, 4, signal routing components 34, 35 of the electrical plug-in connection 33 are sealed off from the outside.

The two electronics units 15 and 31 and/or the interfaces explained above can also be in signal connection with each other wirelessly, for example via the Bluetooth or via the Wifi standard.

Each of the channel sections defined by the channel bodies 11a and 11b is used by one sensor each, namely the ultrasonic sensor 29 on the one hand and the flow sensor 12 on the other hand, as a measuring section for measuring fluid flowing through.

The interface 19 can also be designed as a indicator unit via which digital, optical and/or acoustic signals can be output, for example warning signals. Depending on the data measured via the sensors 12, 29, a limit violation indication can be given via the indicator unit 19, i.e. the indication that predefined measured value limits for the flow rate and/or, if applicable, for detected gas bubble quantities, have been exceeded. In this case, a comparison can be made between a specified target measured value and a measured actual measured value. The indicator unit 19 can emit different tones or tone sequences. The indicator unit 19 can light up in different colors or imitate light sequences. The indicator unit 19 may have an indication vibration module.

The module assembly 1 may further comprise a reusable pump unit 36 for generating a flow of media through the flow channel 10a. The pump unit 36 is shown schematically accommodated in the housing base section 3 in FIG. 3. For example, a peristaltic pump may be used. The use of a micro-pump is also possible.

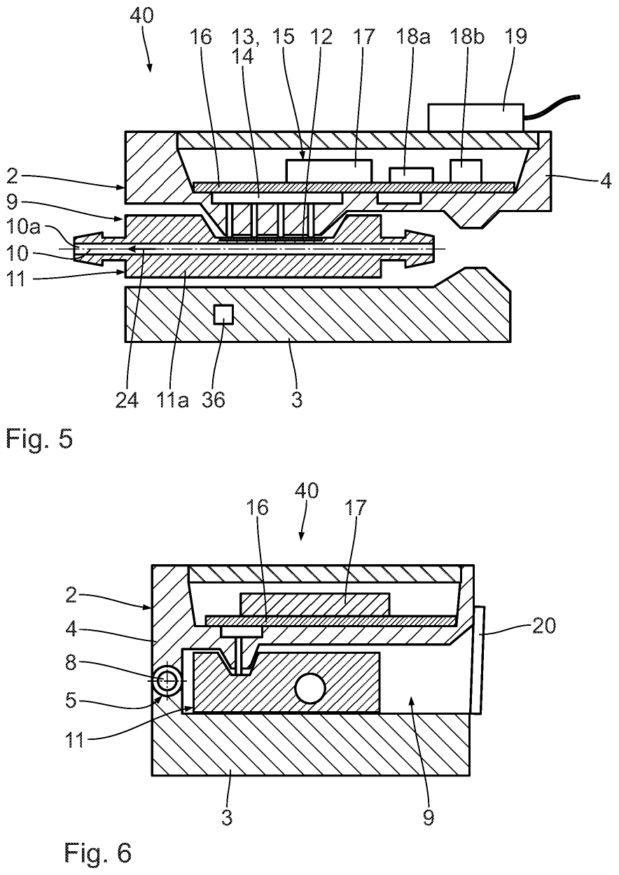

With reference to FIGS. 5 and 6, a further embodiment of a flow sensor module assembly 40 is described below. Components and functions corresponding to those previously explained with respect to FIGS. 1 to 4 bear the same reference signs and will not be discussed again in detail.

The module assembly 40 lacks the base-side electronics unit 31 with the ultrasonic sensor 29. Accordingly, the electrical plug-in connector 33 and also the channel body section 11b can be dispensed with. Otherwise, the module assembly 40 corresponds to the module assembly 1.

The flow sensor module assembly 1 is used as follows:

During the first operation, the two housing sections 3, 4 are folded open around the hinge axis 8 of the hinge 5 and brought into the open position. Now the sensor module 9 is inserted into a complementary recess in the housing base section 3 of the housing module, as shown in FIG. 2. The housing cover section 4 is then brought into the closed position as shown in FIG. 1, wherein the snap-in connection 20 latches and secures the housing cover section 4 in the closed position.

Due to the housing receptacle that is complementary to the shape of the sensor module 9 on the one hand in the housing base section 3 and on the other hand in the housing cover section 4, it results that the housing sections 3, 4 enclose the sensor module 9 in a form-fitting manner. In particular, this prevents the sensor module 9 from rotating about the longitudinal axis 10.

A connection between the sensor module 9 and further fluid-carrying components, in particular tube sections, of the installation for patient monitoring and/or patient care is then established via connection sections 42, 43, which can be designed as Luer connections, at both ends of the channel body 11.

The module assembly 1 is now ready for use and both a flow of fluid through the flow channel 10a and any quantity of bubbles carried along with the fluid can be measured using the sensors 12 and 29, controlled via the respective electronics units 15 and 31. The corresponding measurement data is then recorded via the respective electronics units 15, 31, stored, processed and output to the external device via the respective interfaces. If the module assembly 1 is designed accordingly, a corresponding signal, in particular a warning signal, can also be output via the indicator unit 19.

As soon as a predefined operating time of the single-use sensor module 9 is reached, which can be monitored via a corresponding timer as part of one of the electronics units 15, 31, the need to replace the sensor module 9 is indicated via a corresponding display. By overcoming the snap-in connection 20, the housing cover section 4 is then opened and brought into the open position as shown in FIG. 2. The used sensor module 9 can then be removed and replaced with an unused sensor module or a reconditioned sensor module.

1. A medical flow sensor module assembly (1) as part of an installation for at least one of patient monitoring and patient care,
having at least one single-use sensor module (9), comprising:
a channel body (11) with at least one flow channel (10a),
a flow sensor (12) for measuring a fluid flow through the flow channel (10a); and
a flow sensor interface (13) which is in signal connection with the flow sensor (12),
having at least one reusable housing module (2) for receiving the sensor module (9), comprising:
at least one electronics unit (15) and
an electronics unit interface (14) via which the electronics unit (15) is in signal connection with the flow sensor interface (13),
wherein the reusable housing module (2) has at least two housing sections (3, 4) which enclose the single-use sensor module (9) in a form-fitting manner,
wherein the electronics unit (15) comprises at least one data processing and control component (17, 18, 18a, 18b) for controlling the flow sensor (12) and for receiving, processing and transmitting sensor signals.

2. The module assembly according to claim 1, wherein the electronics unit (15) comprises an interface (19) for signal connection to an external device.

3. The module assembly according to claim 1, wherein the two housing sections (3, 4) each comprise an electronics unit (31, 15).

4. The module assembly according to claim 3, wherein the at least two electronics units (15, 31) are in signal connection with one another via an electrical plug-in connection (33) between the housing sections (4, 3).

5. The module assembly according to claim 1, wherein the two housing sections (3, 4) are hingedly connected to one another by means of a swivel joint (5).

6. The module assembly according to claim 1, wherein the housing module (2) is designed such that the two housing sections (3, 4) are fixedly connected to one another in a folded connection position.

7. The module assembly according to claim 1, comprising at least two sensors (12, 29), one of which is the flow sensor (12) of the sensor module (9).

8. The module assembly according to claim 7, wherein one of the at least two sensors (12, 29) is part of the reusable housing module (2) as a reusable sensor (29).

9. The module assembly according to claim 8, wherein the reusable sensor (29) is designed as an ultrasonic sensor.

10. The module assembly according to claim 1, wherein the channel body (11) is configured to be multi-part such that the flow channel (10a) has a plurality of sequential channel sections which are formed by different channel body sections (11a, 11b).

11. The module assembly according to claim 1, wherein the housing module (2) has an indicator unit.

12. The module assembly according to claim 1, comprising a reusable pump unit (36) for generating a flow of media through the flow channel (10a).
